(19) United States
(12) Patent Application Publication
(10) Pub. No.: US 2019/0183710 A1
(43) Pub. Date: Jun. 20, 2019

(54) MODULAR PATIENT POSITIONING SYSTEM

(71) Applicant: White Surgical Incorporated, Fort Wayne, IN (US)

(72) Inventor: Stephen E. White, Fort Wayne, IN (US)

(73) Assignee: White Surgical Incorporated, Fort Wayne, IN (US)

(21) Appl. No.: 16/282,633

(22) Filed: Feb. 22, 2019

Related U.S. Application Data
(62) Division of application No. 15/015,109, filed on Feb. 3, 2016.

(51) Int. Cl. A61G 13/08; A61G 13/12; A61B 6/04

(52) U.S. Cl. CPC A61G 13/08; A61G 13/121; A61G 13/12; A61B 6/0407; A61G 13/1235

(57) ABSTRACT

Multiple embodiments of a modular patient positioning system for use during surgical procedures are disclosed. In one exemplary embodiment the system comprises two hinged-together pegboards that can be used to support different portions of a patient's body in different planes. Pegs can be further used to retain portions of the patient's body in desired surgical positions. Additionally, a positioning member, such as a pad or cushion may be positioned over several pegs.

### CROSS REFERENCE TO RELATED APPLICATIONS

This is a division of U.S. patent application Ser. No. 15/015,109, entitled “MODULAR PATIENT POSITIONING SYSTEM”, filed Feb. 3, 2016, which is incorporated herein by reference.

### FIELD OF DISCLOSURE

The disclosure is generally directed to the field of modular patient positioning systems (referred to herein as “MPPS”). In particular, the disclosure is directed to the field of MPPSs for use in orthopedic surgery and conveniently positioning or extending different body portions to be operated upon in different positions. The system of the current disclosure allows surgeons and operating room staff to use a reduced amount of hardware in combination with a foundational device to position, support, and improve the results of the surgical procedure. Moreover, small movements that can cause joint misalignment are preventable with the system. The modularity of the system also allows one system to be used for posterior or anterior hip surgery, knee surgery, extremity surgery, as well as a sit-up position for neck or shoulder surgery.

### BRIEF DESCRIPTION OF THE DRAWINGS

FIG. 1A shows a first exemplary embodiment of an MPPS, which comprises two separate pegboards and in a first configuration. FIG. 1B shows another exemplary embodiment of an MPPS that comprises one pegboard.

FIG. 2 shows the first exemplary embodiment of the MPPS of FIG. 1A in a second configuration.

FIG. 3A, FIG. 3B, FIG. 3C show various exemplary ways that the MPPS of FIG. 1A may be used during surgery.

FIG. 4 shows how when the MPPS of FIG. 1A is in the second configuration, the patient's upper body is in a raised position that makes surgery on the upper portion of the patient's upper body easier.

FIG. 5A and FIG. 5B show other exemplary embodiments of an MPPS; in these exemplary embodiments, pegs are shown for retaining a portion of a patient's body in an operating position. The embodiment of FIG. 5A is wider than the embodiment of FIG. 5B.

FIG. 6 shows another embodiment of the MPPS; in this exemplary embodiment, the MPPS is for supporting a first part of the patient's upper body and providing unobstructed access to a second part of the patient's upper body, on which surgery is to be performed.

FIG. 7A, FIG. 7B are rear views, showing how the exemplary embodiment of FIG. 6 allows unobstructed access to either of the left or right side parts of the patient's upper body, on which surgery is to be performed.

FIG. 8 shows another exemplary embodiment of the MPPS; in this exemplary embodiment, an arm pegboard extends outwardly beyond side edges of the second pegboard and a surgical table.

FIG. 9A, FIG. 9B, FIG. 9C show different exemplary ways in which additional pegboards, such as the arm pegboards of FIG. 8, may be connected to the rest of the MPPS.

FIG. 10A and FIG. 10B show examples of performing arm surgeries with the exemplary embodiments of FIG. 1A and FIG. 8 respectively.

FIG. 11A, FIG. 11B, FIG. 11C show additional exemplary embodiments of the MPPS; in these exemplary embodiments, the MPPS includes an x-ray facilitating cut-out.

FIG. 12A, FIG. 12B, and FIG. 12C show another exemplary embodiment of the MPPS; in this exemplary embodiment, a generic positioning member for retaining a portion of the patient's body that is to be operated on in a desired surgical position using pegs as a supporting/holding mechanism is included.

FIG. 13 shows the exemplary generic positioning member of FIGS. 12A, 12B, and 12C, in use positioning a patient body in a laterally upright configuration.

FIG. 14A-14C, show other exemplary embodiments of an MPPS, these exemplary embodiments including positioning members for use on surgery on different specific body parts.

FIG. 15A and FIG. 15B show an exemplary embodiment of an MPPS for facilitating surgery on a patient's neck and/or shoulder area.

FIG. 16A and FIG. 16B show additional exemplary embodiment of an MPPS.

FIG. 17 shows the exemplary embodiment of the MPPS of FIG. 16 in a configuration for surgery on a portion of a patient's lower body and positioned on a surgical table, similar to FIG. 3A, FIG. 3B, and FIG. 3C.

FIG. 18A, FIG. 18B, FIG. 18C, FIG. 18D, FIG. 18E show the exemplary embodiment of the MPPS of FIG. 16 in positions for conducting surgery on one of a patient's legs (FIG. 18A), a patient's knee (FIG. 18B), and/or both of a patient's legs (FIG. 18C). FIGS. 18C and 18D show the use of a leg positioner for flexing, e.g., a knee, during surgery thereon.

FIG. 19A, FIG. 19B, and FIG. 19C, show the exemplary embodiments of FIGS. 18A, 18B, and 18C, respectively in side view.

FIG. 20 shows the exemplary embodiment of the MPPS of FIG. 16A in a configuration for performing surgery on an upper portion of a patient's body.

FIG. 21A and FIG. 21B are rear views similar to those of FIG. 7A and FIG. 7B, respectively, where different portions of the patient's upper body are supported and different portions of the patient's upper body are unobstructed for surgery thereon.

FIG. 22 is a rear view, similar to the rear views of FIG. 21A and FIG. 21B, but showing where the MPPS is configured to support substantially the entire upper body.

### DETAILED DESCRIPTION OF THE INVENTION

It will be appreciated that for simplicity and clarity of illustration, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure. Similar reference numerals are used to refer to structures similar to the various exemplary embodiments.

The embodiments shown and described above are only examples. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in decorative and structural matters of shape, size and arrangement of the parts within the principles of the present disclosure up to, and including the full extent established by the broad general meaning of the terms used in the claims. It will therefore be appreciated that the embodiments described above may be modified within the scope of the claims.

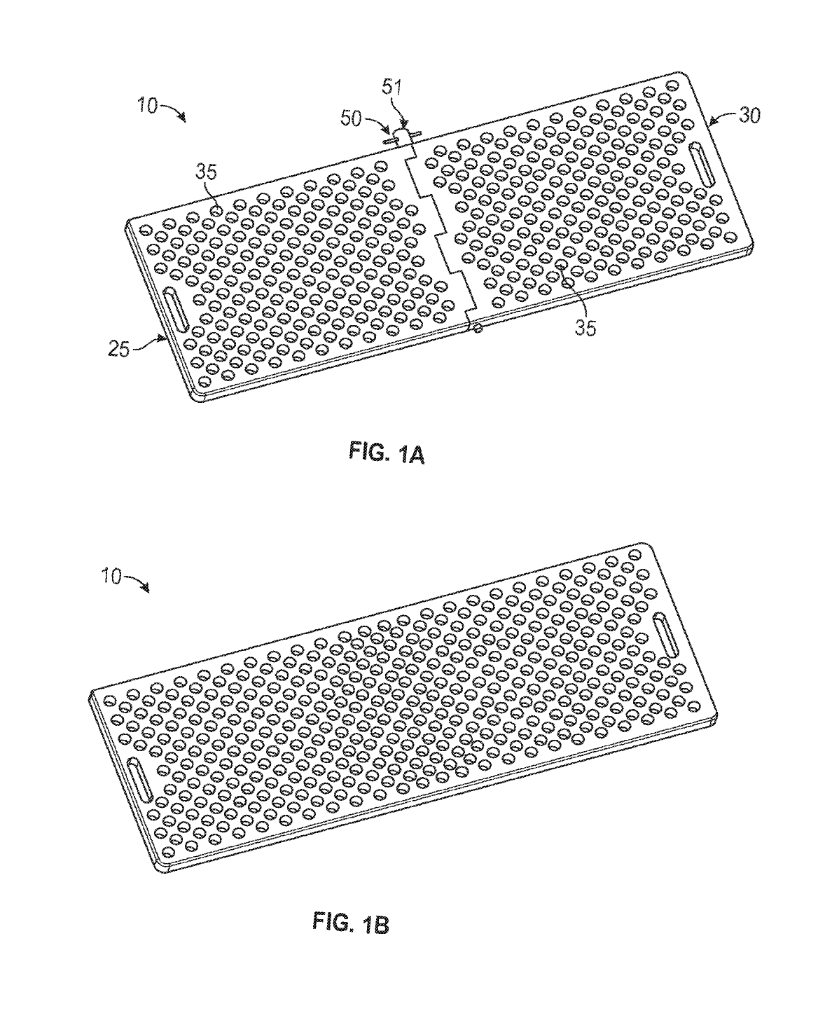

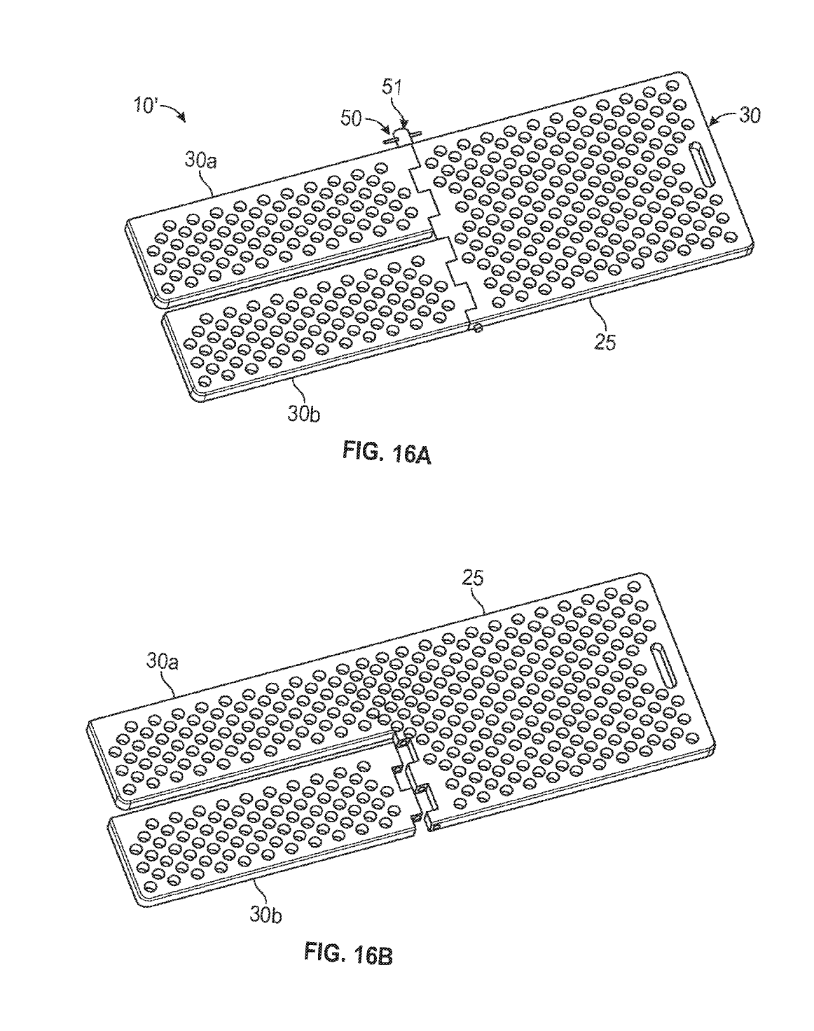

FIG. 1A shows a first exemplary embodiment of a modular patient positioning system (“MPPS”) 10 and in a first configuration. This exemplary embodiment of the MPPS 10 includes at least first pegboard 25 and second pegboard 30. Typically, though not necessarily, the first pegboard 25 and the second pegboard 30 are generally the same size. The first pegboard 25 and the second pegboard 30 have peg holes 35 therein, for reasons discussed below. The at least first pegboard 25 and second pegboard 30 may be connected together, such that the first pegboard 25 and second pegboard 30 can be rotated relative to each other between different configurations. In the exemplary embodiment of the MPPS of FIG. 1A; the first pegboard 25 and second pegboard 30 are connected together by a hinge 50. Hinge 50 may comprise a removable hinge pin 51. However, as exemplified in FIG. 16B, hinges are not necessary.

In a first configuration, as shown in FIG. 1A, the first pegboard 25 and second pegboard 30 lie along a common plane. In FIG. 1B, the MPPS comprises a single pegboard.

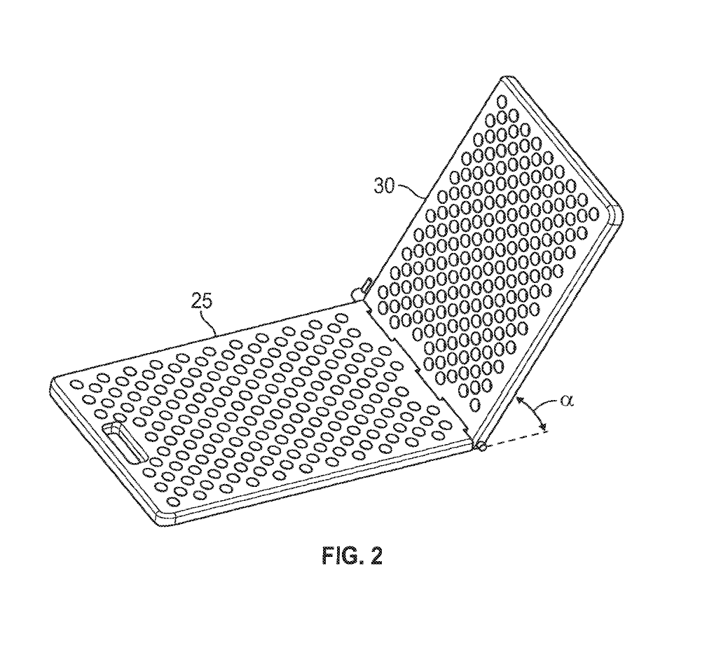

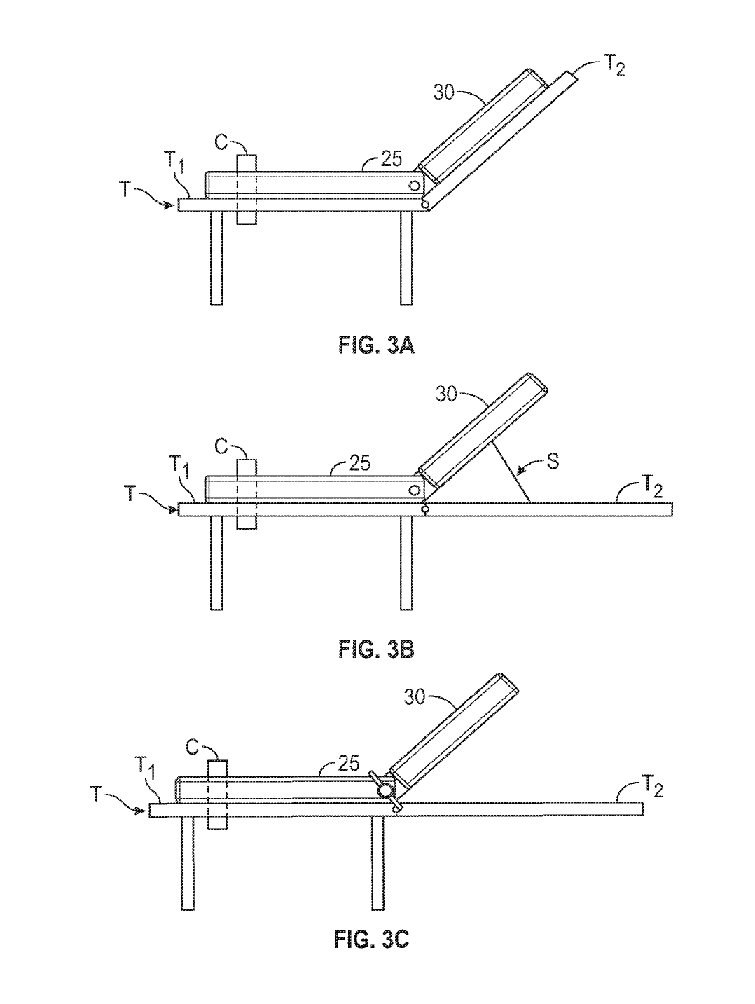

In a second configuration, as shown in FIG. 2, the first pegboard 25 and second pegboard 30 lie along different planes at an angle α. The second configuration allows for different parts of a patient's body to be positioned and supported in different planes. The second configuration usually includes one pegboard lying flat (e.g. substantially horizontally) or parallel to a top surface of a surgical table and the other pegboard being at a positive or negative angle α relative to the flat pegboard (see discussions of FIGS. 3A-3C below). For ease of reference and not intended to be limiting in any way, this disclosure will refer to the “second pegboard” as the pegboard that is raised or lowered, as discussed further below.

FIGS. 3A-3C show various exemplary ways by which the second pegboard 30 can be supported in a raised position relative to the first pegboard 25.

As shown in FIG. 3A, the MPPS 10 may be supported on (by) a surgical/operating table T (schematically shown). First pegboard 25 may be clamped to a first, typically fixed-positioned part T1 of the surgical table T by a clamp C. The second pegboard 30 is supported in the raised position by a rotatable portion T2 of the surgical table T and may or may not be clamped to rotatable portion T2.

As shown in FIG. 3B, the MPPS 10 may be supported on (by) a surgical/operating table T (schematically shown). First pegboard 25 may be clamped to a first, typically fixed-positioned part T1 of the surgical table T by a clamp C. The second pegboard 30 is moved into the raised position and supported in the raised position by a support member S, such as a bar, frame, or wedge, between the top of the surgical table and an underside of the second pegboard 30.

As shown in FIG. 3C, the MPPS 10 may be supported on (by) a surgical/operating table T (schematically shown). First pegboard 25 may be clamped to a first, typically fixed-positioned part T1 of the surgical table T by a clamp C. The second pegboard 30 is moved into the raised position and supported in the raised position by hinge pin 51 comprising a locking hinge pin.

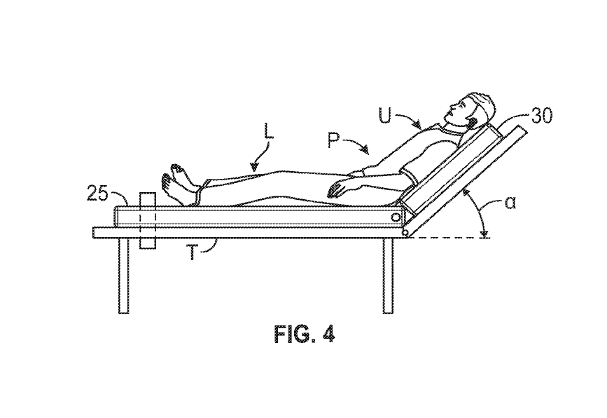

The ability for the first pegboard 25 and second pegboard 30 to rotate relative to each other and receive pads/cushions and pegs makes it easier for a doctor to position different patient's in different body positions using pegs, pegboards, and pads to perform surgery on, for example, upper portions U of a patient's body P. For example, as shown in FIG. 4, when the MPPS is in the second configuration: the first pegboard 25 lies substantially flat on a surgical table T and the second pegboard 30 is rotated upward and away from the plane of the first pegboard 25. In this second configuration, the patient's lower body L lies generally flat on the first pegboard 25 substantially parallel with the surgical table T and the patient's upper body U is supported at a raised angle α above the plane of the first pegboard, to present the patient's upper body U in a raised position that makes surgery on an upper portion U of the patient's upper body U easier.

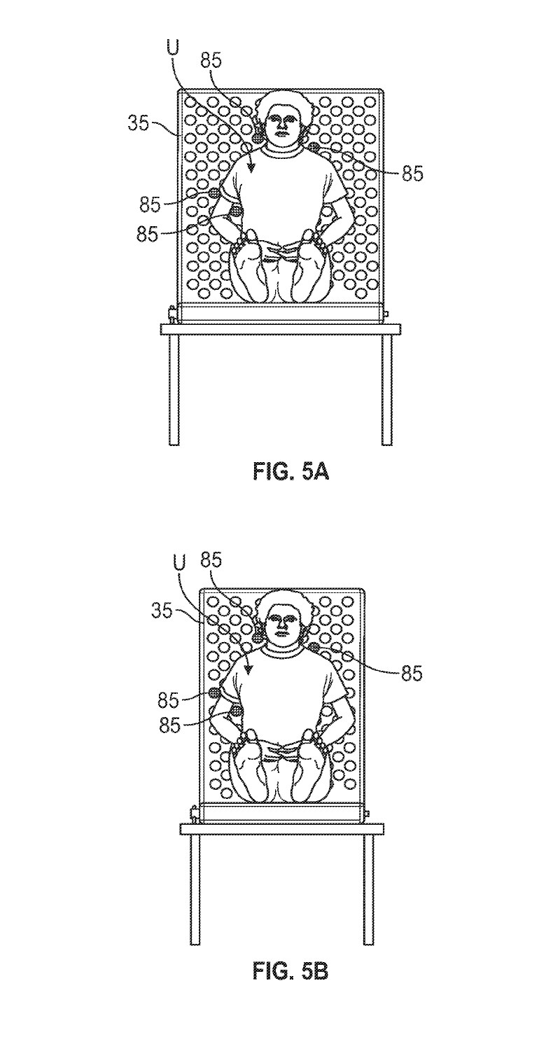

Thus, when the part of the patient's upper body U to be operated on comprises a shoulder, a doctor may use pads/cushions and pegs (see discussion about FIGS. 5B, 5B, below) to specifically position different patient's in different body positions using pegs. This reduces doctor fatigue during the surgical procedure and maintains desired patient positioning.

The angle α at which the second pegboard supports the patient's upper body relative to the plane of the first pegboard can vary, but generally depends on the heights of the surgical table and the doctor, so that the area of the patient to be operated on is at a position where the surgeon need not lean over the patient, stand in any awkward postures, etc.

As shown in FIG. 5A and FIG. 5B, one or more pegs 85 may be inserted into peg holes 35 and protrude outwardly to retain a portion of the patient's upper body U in a desired surgical position.

As shown in the exemplary embodiment of FIG. 1, the first pegboard 25 and second pegboard 30 are generally identical in structure, allowing ease of installation and replacement of broken parts. However, the first pegboard 25 and second pegboard 30 need not be identical in structure.

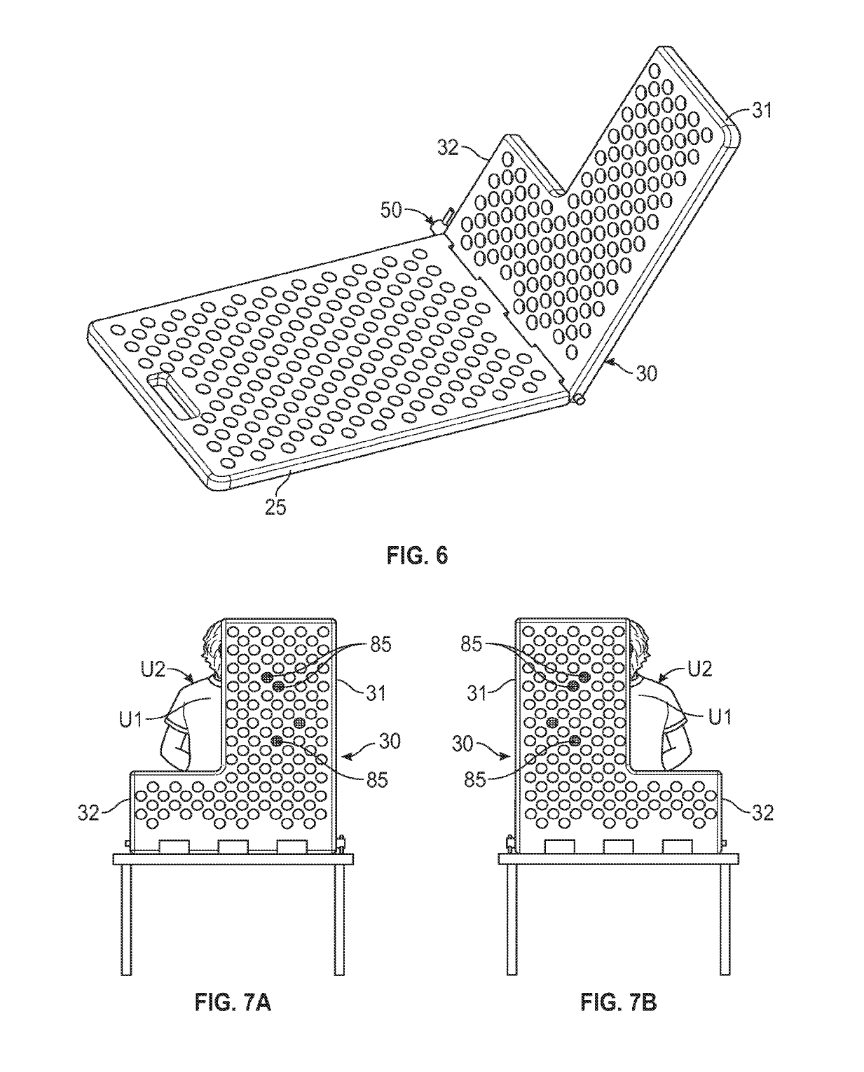

For example, as shown in FIG. 6, the second pegboard 30 is L-shaped having a narrow portion 31 and a wide portion 32. Wide portion 32 includes the hinge portion of second pegboard 30. The first pegboard 25 is generally rectangular and equal in width to the wide portion 32 of the second pegboard 30. Thus, the hinge portion of wide portion 32 of the second pegboard 30 can be conveniently connected to the hinge portion of the first pegboard 25 by the hinge pin 51.

As shown in the rear views of FIG. 7A and FIG. 7B, the narrow portion 31 of the second pegboard 30 supports a first part of the patient's upper body U1 and provides unobstructed access to a second part of the patient's upper body U2, on which surgery is to be performed. For example, as shown in rear view FIG. 7A, the patient's left shoulder is supported by narrow portion 31 of the second pegboard 30 and the patient's right shoulder is unobstructed for surgery. As shown in rear view FIG. 7B, the second, L-shaped pegboard may be disconnected from the first pegboard, reversed in configuration, and then reconnected to the first pegboard, whereby the second, L-shaped pegboard may be positioned to provide unobstructed access to the left side of the patient's upper body.

MPPS 10 can be used to raise or lower a patient's legs for surgery thereon, rather than the upper body by merely reversing the positioning of the first pegboard portion and the second pegboard portion. This is described in more detail below. For example, as described below, raising or lowering the second pegboard portion can be used to flex or relax a patient's knee or hip.

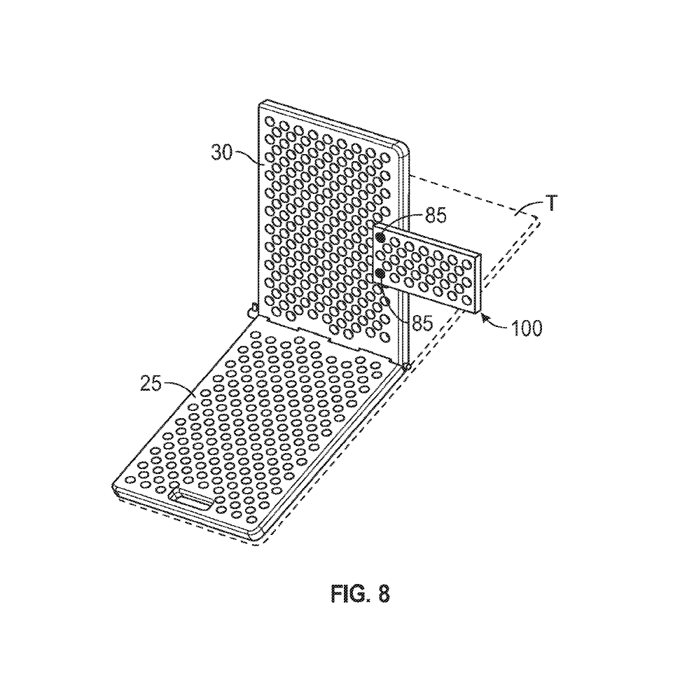

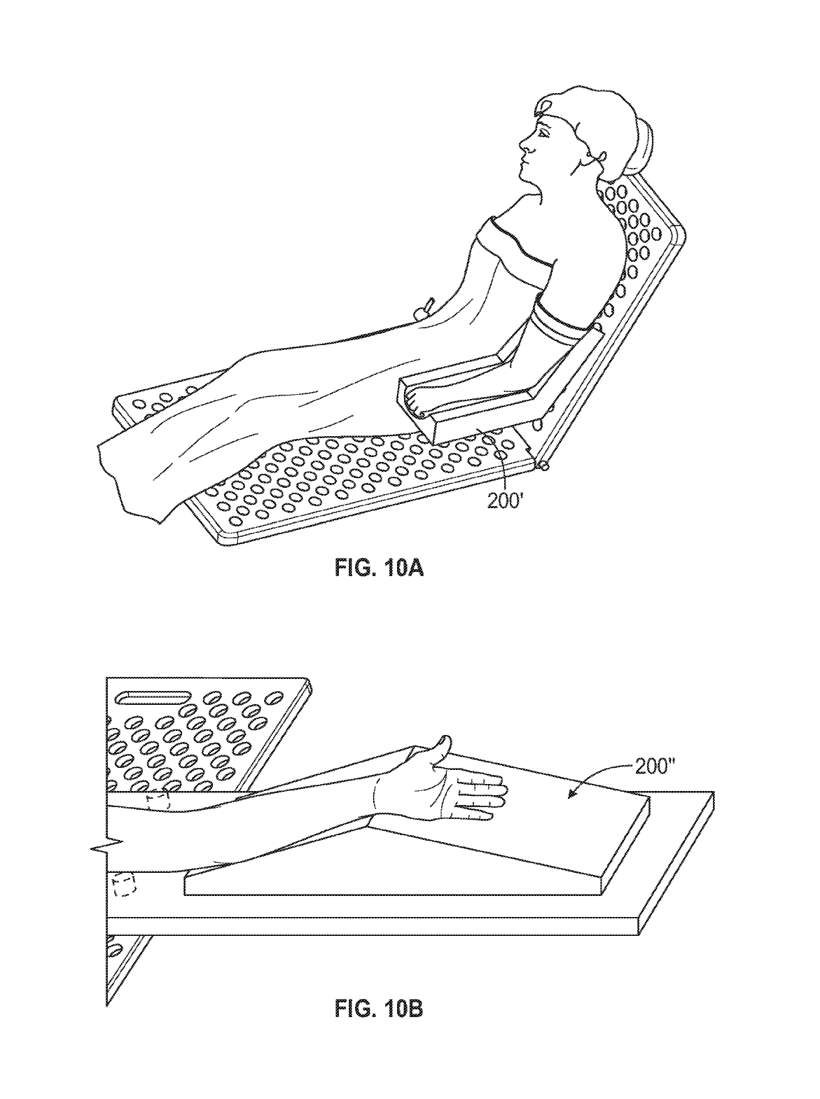

In another exemplary embodiment, as shown in FIG. 8, the MPPS 10 may include additional pegboards for supporting various other body portions. For example, in FIG. 8 an arm pegboard 100 is shown that can be connected to the second pegboard 30. In FIG. 8, the arm pegboard 100 extends outwardly beyond side edges of the second pegboard 30 and surgical table T, and may support an arm positioning member 200″ and a patient's arm for being operated on as shown in FIG. 10B. As shown in FIG. 10A, an arm positioning member 200′ that can be connected to the pegboard by pegs can support the patient's arm outwardly.

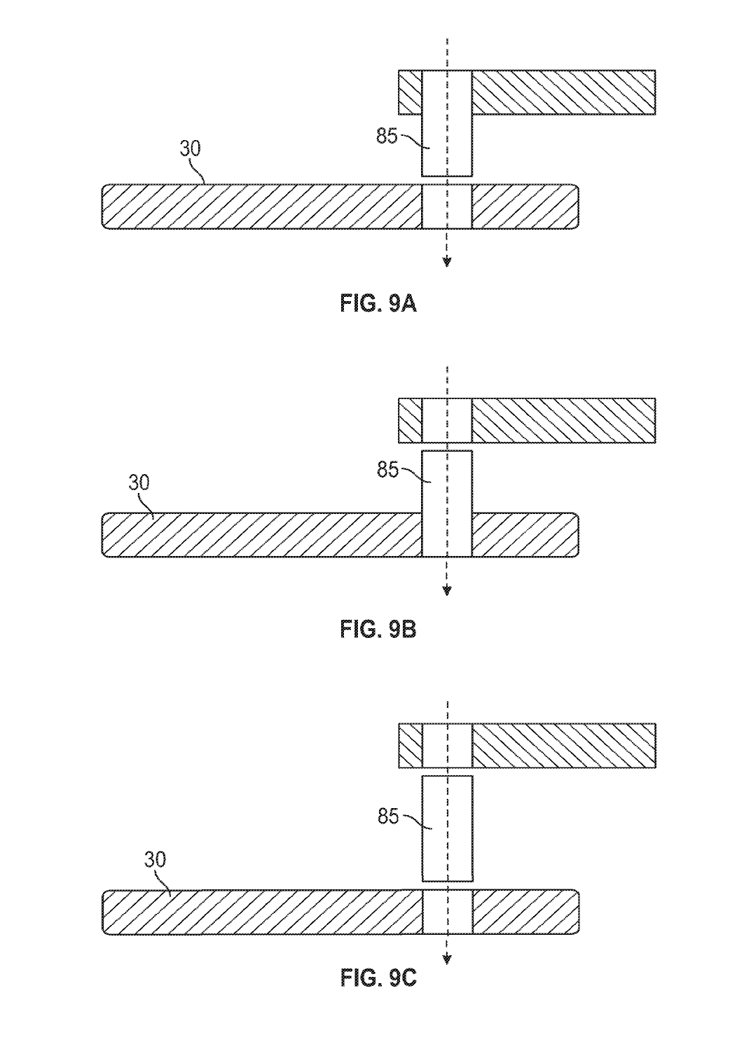

As shown in FIG. 9A, pegs 85 may be permanently affixed to the arm (or other additional) pegboard to be received in the peg holes 35 of the first pegboard 25 or second pegboard 30. As shown in FIG. 9B, pegs 85 may be permanently affixed to the first pegboard 25 or second pegboard 30 to be received in the peg holes 35 of the additional pegboard 100. Yet further, as shown in FIG. 9C, the pegs 85 may be separate elements that are received in the peg holes 35 of the first pegboard 25 or second pegboard 30 and peg holes 35 in the additional pegboard 100.

As shown in FIG. 10A, when a first exemplary arm pegboard with a first exemplary positioning member 200′ does not extend beyond the edges of the second pegboard 30 and the surgical table T, a patient's arm may be supported/stabilized in a first operating position while surgery on the supported arm or opposite shoulder is performed. As shown in FIG. 10B, when a second exemplary arm pegboard with a second exemplary positioning member 200″ extends beyond the edges of the second pegboard 30 and the surgical table T, the patient's arm may be supported in a different operating position and/or flexed or relaxed. These multiple positions allow for proper positioning and support on a patient's arm/wrist/hand during surgery thereon. For sake of convenience, as used in the disclosure and claims “arm” shall be construed to be any body portion below the shoulder, including but not limited to, upper arm, elbow, lower arm, wrist, hand, or fingers. Similarly “leg” shall be construed to cover any body portion between the tips of the toes and the hip/pelvis.

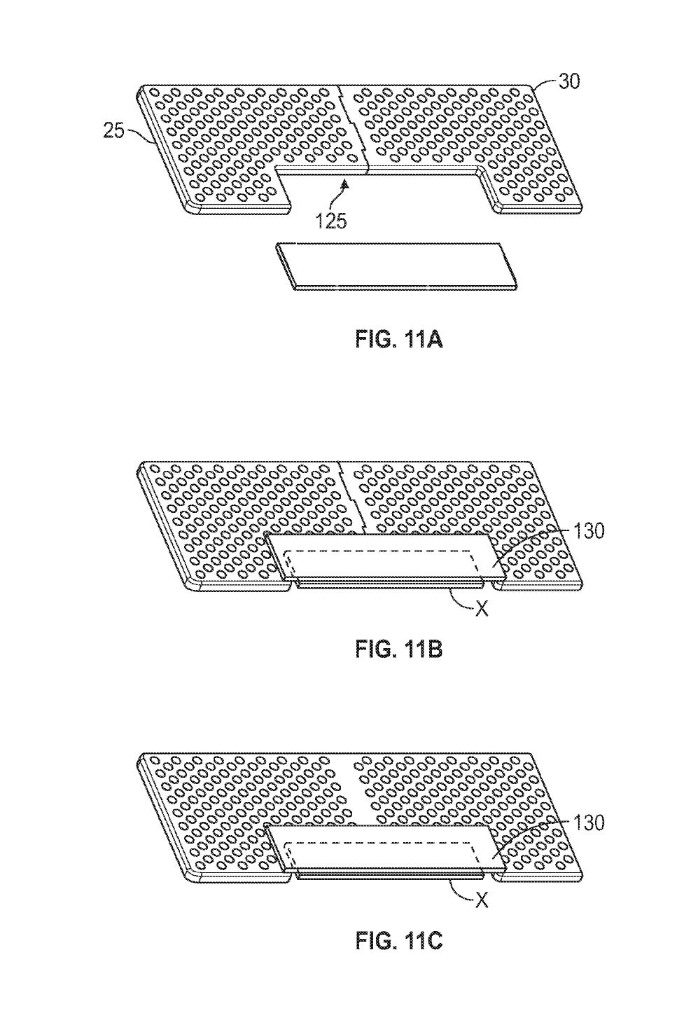

Another feature that the MPPS may include is schematically shown in FIGS. 11A, 11B, 11C, wherein at least one of the pegboards 25, 30 has a cut-out 125 therein. This cutout allows an x-ray film (or cassette) X to be inserted into the area of the cut-out 125 for taking an unobstructed x-ray of the patient, i.e., without the blocking of the dense solid portions of the pegboard. As shown in FIG. 11B, a thin radiolucent cover 130 may be positioned over the cut-out 125 for receiving the x-ray film X thereunder while supporting the patient's body so there is no contact between the x-ray film X and the patient's skin. In FIG. 11C, the single pegboard of FIG. 1B is provided with cutout 125.

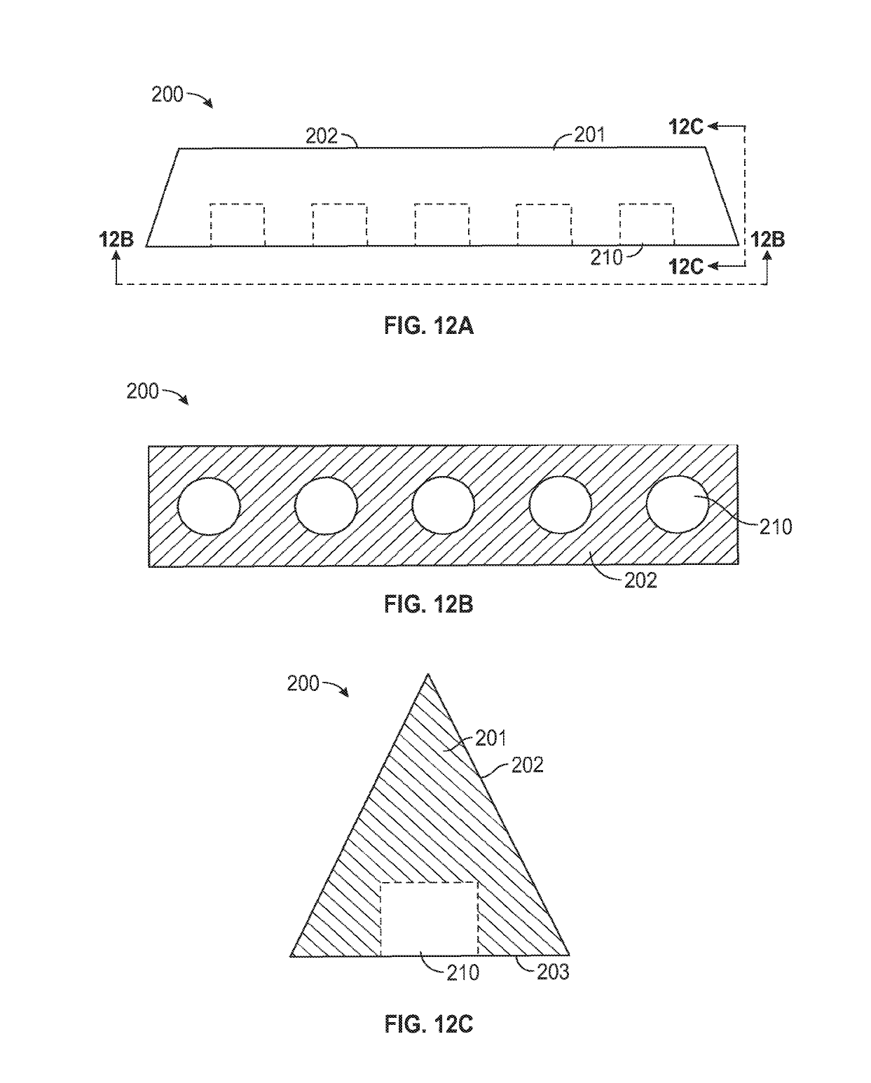

Another feature that the MPPS may include is shown in FIG. 12A, FIG. 12B, FIG. 12C. This feature includes a generic patient positioning member 200 used to cover at least two pegs 85 being used to retain the portion of the patient's upper body that is to be operated on in a desired surgical position. Conventionally, in prior art methods, one peg, in combination with tape, is used to position the pad and retain it in its proper position. As shown in FIG. 12A, FIG. 12B, FIG. 12C the positioning member 200 comprises a positioning pad 201 (also known in the art as cushion or pillow). The positioning pad 201 has an upper portion 202 and a lower portion 203; the upper portion 202 for contacting a portion of the patient's body; and the lower portion 203 having a plurality of blind holes 210 for receiving pegs 85.

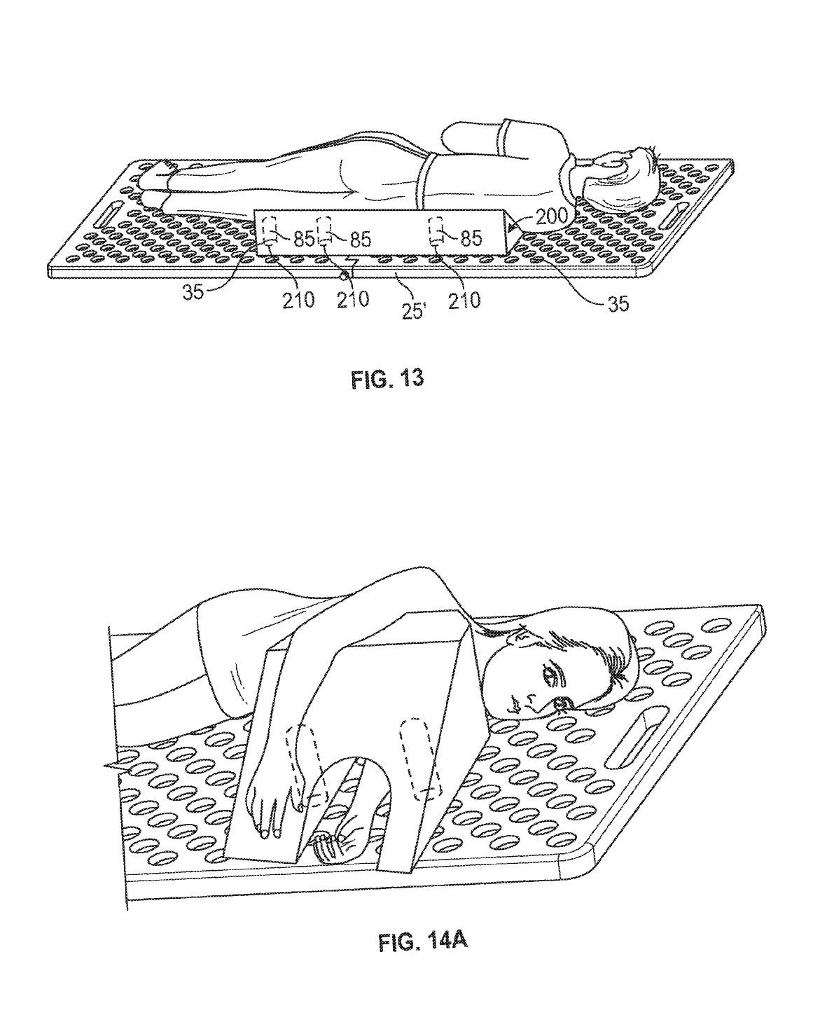

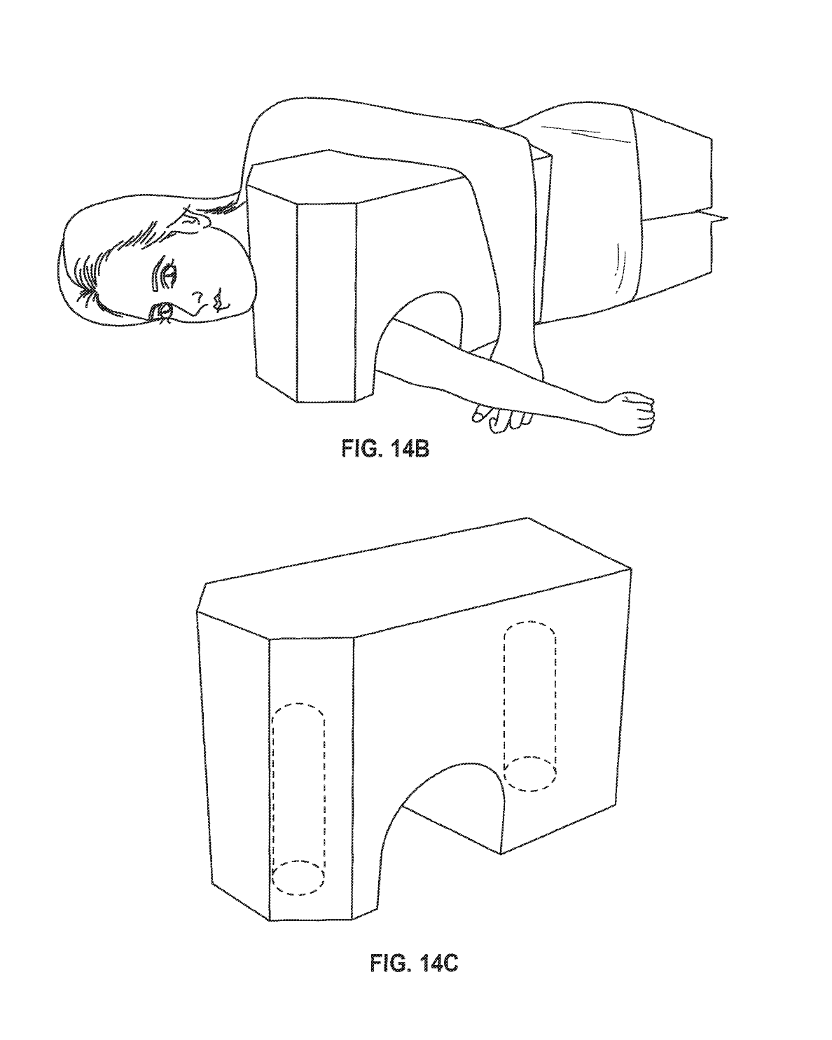

The positioning member 200 can be used either in combination with an MPPS having a first pegboard 25 and second pegboard 30 or with a conventional full-sized pegboard (FIG. 1B). For example, as shown in FIG. 13, the MPPS may include at least one pegboard 25′ for positioning on a surgical table and supporting a portion of a patient. Positioning member 200 aligns a portion of a patient's body in a surgical position. The positioning member has a plurality of holes 210 in a lower portion thereof. A plurality of pegs 85 are positioned in peg holes 35 at locations where it is desired to position the positioning member 200. The positioning member 200 is then placed over the plurality of pegs 85, by the pegs 85 being received in the holes 210 in the lower portion of the positioning member 200. Conventionally, in prior art methods, positioning pads are fixed with tape to the table or pegboard to retain it in its proper position, or pads are wrapped around a single peg when the patient is fixed in a lateral position. FIG. 14A, FIG. 14B, and FIG. 14C show other exemplary configurations for positioning member 200, useful with different and specific surgical procedures on various body parts.

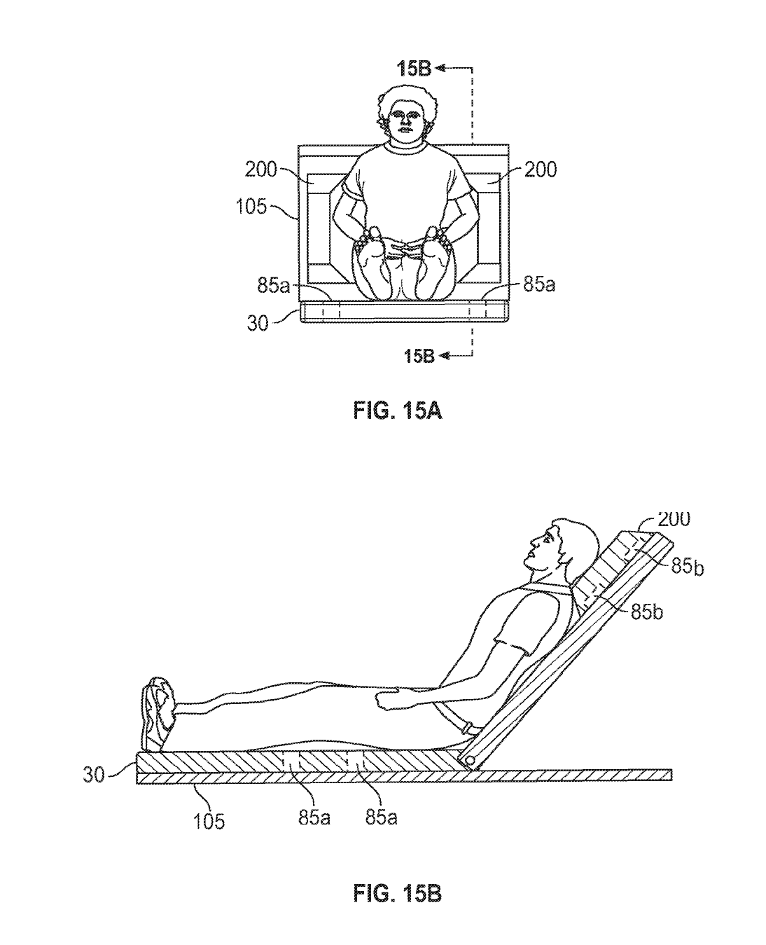

FIG. 15A and FIG. 15B show an exemplary embodiment of an MPPS for facilitating surgery on a patient's neck and/or shoulder areas. In this exemplary embodiment, a first set of pegs 85a are used to attach another (e.g., neck) pegboard 105 to the upper portion/free end of the second pegboard 30. A second set of pegs 85b protrude from the neck pegboard 105 on each side of the patient's neck. Finally, positioning members 200 are placed over pegs 85b, as previously described. This configuration allows for a neck/shoulder positioning that is comfortable to both the doctor and patient.

FIG. 16A shows another exemplary embodiment of an MPPS. In FIG. 16A, MPPS 10′ is similar to the MPPS 10 of FIG. 1. The difference between MPPS 10′ and MPPS 10 is that MPPS 10′ has one of pegboards 25, 30, divided into two, separately rotatable pegboard parts, e.g., 30a, 30b. Furthermore, in FIG. 16B, pegboard part 30b is not hinged to the rest of the pegboard 25. In FIG. 16B, pegboard parts 30b may be clamped to the surgical table T in manners previously described. Typically pegboard parts 30a, 30b will each have substantially the same width, and their combined width will be substantially equal to the width of the other pegboard, e.g., 25.

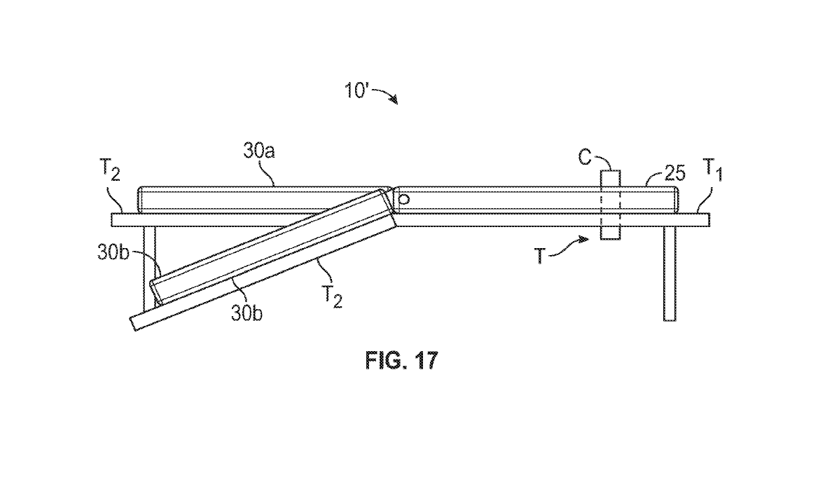

FIG. 17 shows the exemplary embodiment of the MPPS of FIG. 16A in a configuration for surgery on a portion of a patient's lower body and positioned on a surgical table, similar to FIG. 3A, FIG. 3B, and FIG. 3C. In FIG. 17, MPPS 10′ is supported on (by) a surgical table T (schematically shown). First pegboard 25 may be clamped to a first, typically fixed-position part T1 of the surgical table T by a clamp C. The second pegboard portions 30a, 30b, are supported in a raised or lowered position by a rotatable portion T2 of the surgical table T and may or may not be clamped to rotatable portion T2.

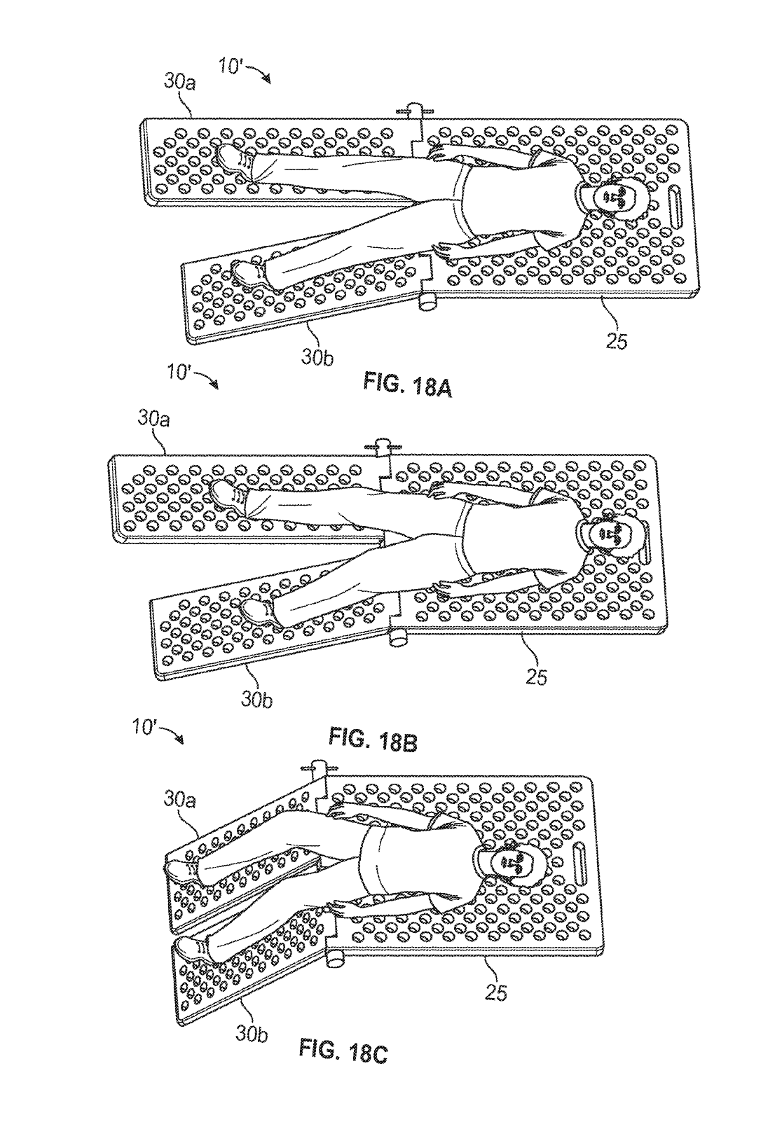

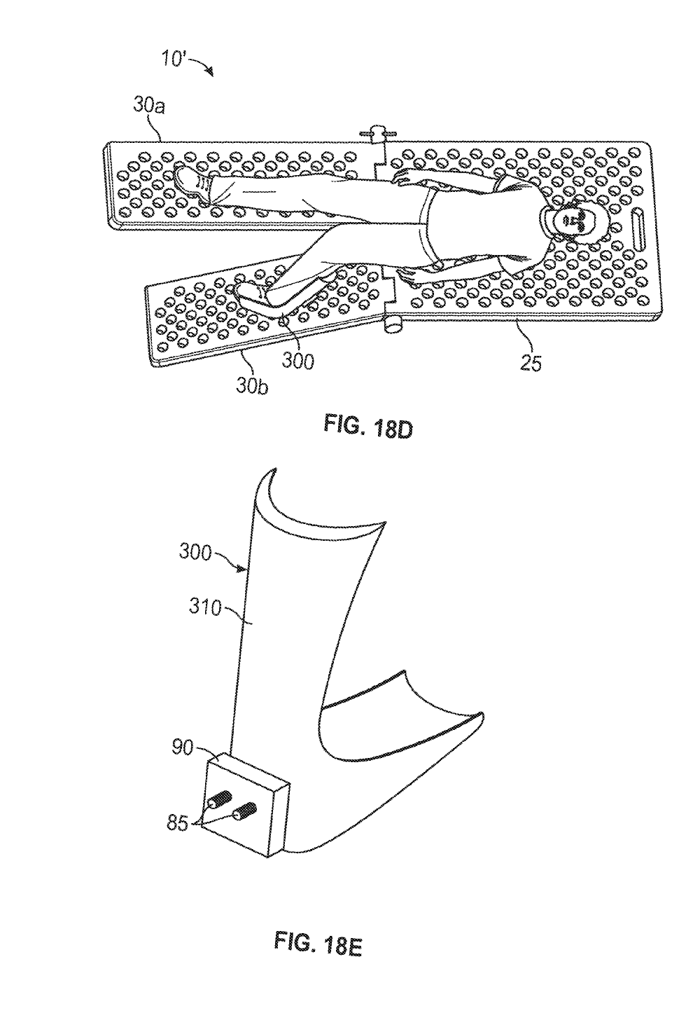

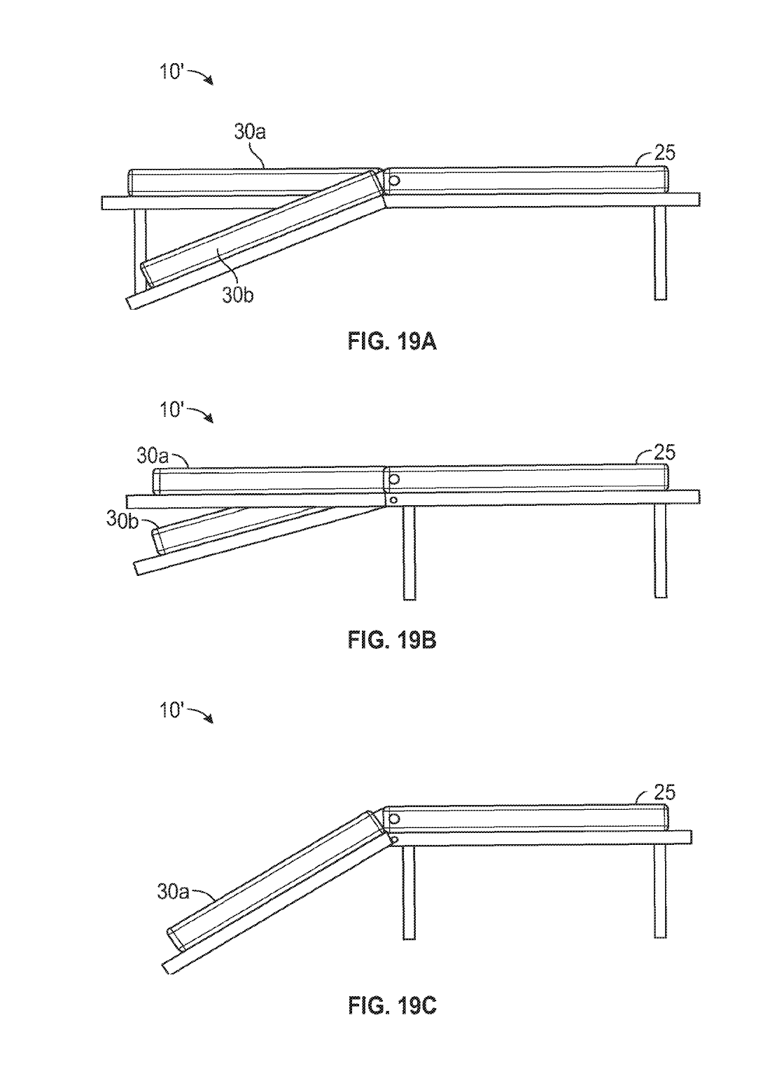

FIG. 18A, FIG. 18B, FIG. 18C each schematically show the exemplary embodiment of the MPPS 10′ of FIG. 16 in positions for conducting surgery on one of a patient's legs (FIG. 18A), a patient's knee (FIG. 18B), and/or both of a patient's legs (FIG. 18C). Note that this functionality is useful because often, when a leg is being operated on, it may be angled in an up or down positioning to check rotation, provide visibility to all portions of the joint, and extend or relax muscles and ligaments. For clarity, FIG. 19A, FIG. 19B, and FIG. 19C, show the exemplary embodiments of FIGS. 18A, 18B, and 18C, respectively in side view.

In FIG. 18A, the patient's hip is generally aligned with the pivot axis between the first pegboard and the second pegboard. Therefore, pivoting of the pegboards cause either tensioning or extension (extend or relax) of the hip muscles. In FIG. 18B, the patient's knee is generally aligned with the pivot axis between the first pegboard and the second pegboard. Therefore, pivoting of the pegboards cause either tensioning or extension (extend or relax) of the knee muscles. To further accurately position a knee during surgery, a leg positioner 300 may be used. This leg positioner structure is shown in FIG. 18D and FIG. 18E. Leg positioner 300 includes a boot 310 for receiving the foot and lower portion of the leg whose knee is being operated on. Pegs 85 are attached to the boot so that the boot can be fixed to pegboard 30b in a desired position that provides the desired flexion on the knee, when pegboard 30b is adjusted. While not necessary, a stabilization block 90 may also be provided to ease the shearing forces on pegs 85. While not shown, it is possible that a surgical boot having a peg attached thereto can be used to attach the foot to the pegboard to assist in flexing or extending the knee. The leg positioner 300 and arm positioner 200′ (FIG. 10A) can be attached to the pegboards in substantially the same ways.

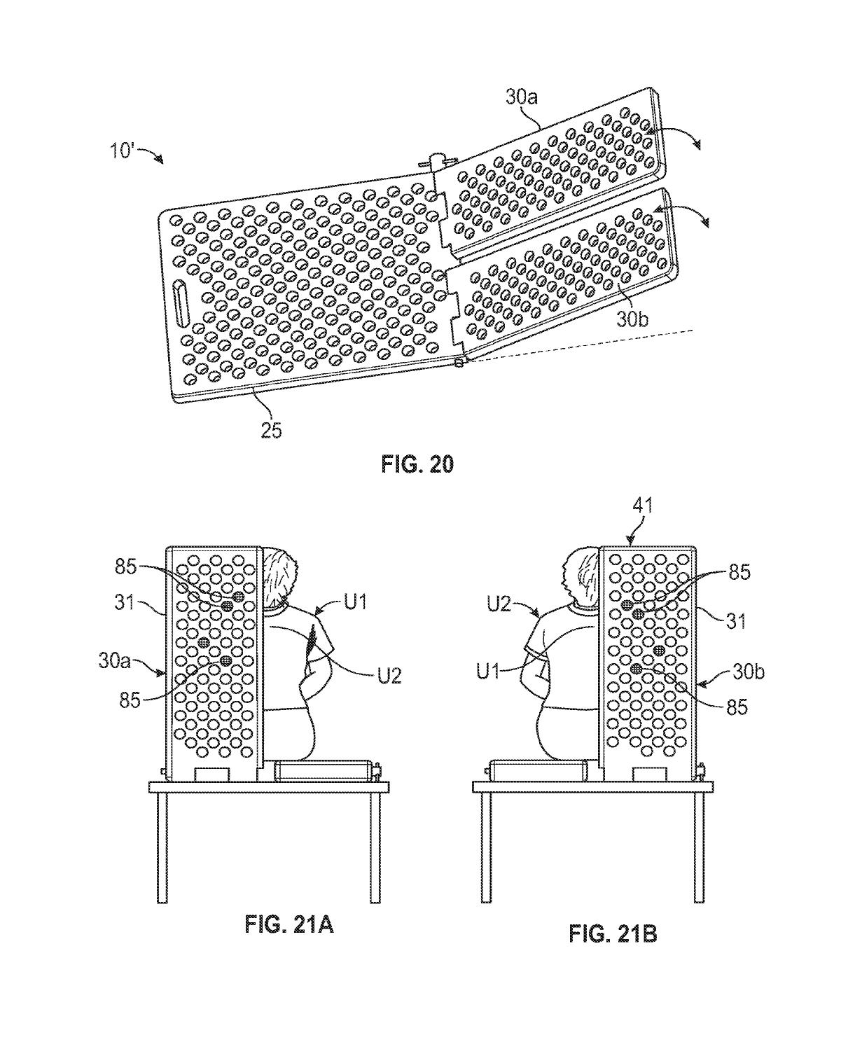

FIG. 20 shows the exemplary embodiment of the MPPS of FIG. 16 in a configuration for performing surgery on an upper portion of a patient's body. The details of this embodiment are generally the same as those of FIG. 1, except for the fact that second pegboard 25 is formed by two second pegboards 30a, 30b.

FIG. 21A and FIG. 21B are rear views similar to those of FIG. 7A and FIG. 7B, respectively, where different portions of the patient's upper body U2 are supported and different portions of the patient's upper body U1 are unobstructed for surgery thereon. Reference to FIG. 7A and FIG. 7B will generally inform the reader of the features and functionality of FIG. 21A and FIG. 21B.

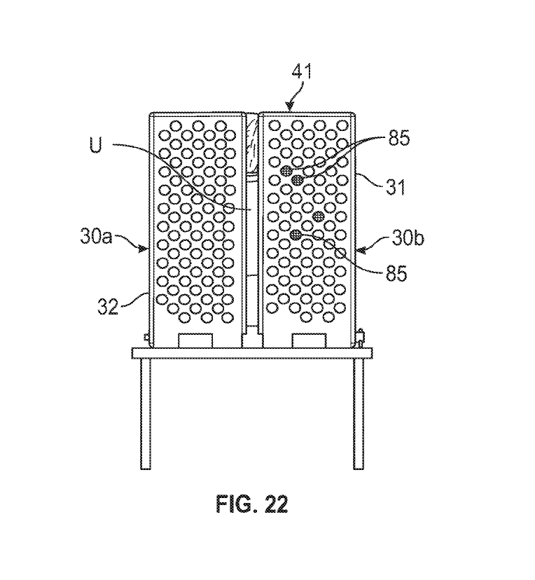

FIG. 22 is a rear view, similar to the rear views of FIG. 21A and FIG. 21B, but showing where MPPS 10′ is configured to support substantially the entire upper body U. Again, reference to FIG. 7A and FIG. 7B will generally inform the reader of the features and functionality of FIG. 22.

The embodiments shown and described above are only examples. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in decorative and structural matters of shape, size and arrangement of the parts within the principles of the present disclosure up to, and including the full extent established by the broad general meaning of the terms used in the claims. It will therefore be appreciated that the embodiments described above may be modified within the scope of the claims.

What is claimed is:

1. A modular patient positioning system, comprising:
a first pegboard and a second pegboard each having a plurality of pegboard holes, the first pegboard and second pegboard are connected together by a hinge and movable about said hinge; wherein: in a first configuration, the first pegboard and second pegboard lie along a common plane; in a second configuration, the first pegboard and second pegboard lie along different planes,
at least one of said first and second pegboards has a cut-out therein, said cutout including means for holding an x-ray film container to provide for an x-ray to be taken unobstructed from either the first or second pegboard;
said hinge including a locking means to selectively prevent movement between said first and second pegboard;
a leg positioner including a boot portion adapted to receive a foot and a lower portion of a leg; the boot portion further comprising pegs for being received in the at least one of said pegboard,
a third pegboard connectable to one of said first and second pegboards and adapted to support a body portion thereon at a position extending outwardly of said first and second pegboards;
at least one peg configured to be positioned within one or more of said plurality of peg holes and adapted to form a support; and
a positioning pad configured to cover said peg and support a patient in a desired position.

2. The modular patient positioning system of claim 1, wherein the second pegboard is L-shaped having a narrow portion and a wide portion and is configured to be removably connected to said first pegboard by said hinge such that the wide portion and the narrow portion of the second pegboard can be selectively connected to said first pegboard by said hinge.

3. A modular patient positioning system, comprising:
a first pegboard and a second pegboard each having a plurality of pegboard holes, the first pegboard and second pegboard are connected together by a hinge and movable about said hinge; wherein: in a first configuration, the first pegboard and second pegboard lie along a common plane; in a second configuration, the first pegboard and second pegboard lie along different planes,
at least one of said first and second pegboards has a cut-out therein, said cutout including means for holding an x-ray film container to provide for an x-ray to be taken unobstructed from either the first or second pegboard.

4. The modular patient positioning system of claim 3, wherein said hinge including a locking means to selectively prevent movement between said first and second pegboard.

5. The modular patient positioning system of claim 4, further including a third pegboard connected to one of said first and second pegboards and adapted to support a body portion thereon at a position extending outwardly of said first and second pegboards.

6. The modular patient positioning system of claim 4, further including
at least one peg configured to be positioned within one or more of said plurality of peg holes and adapted to form a support; and
a positioning pad configured to cover said peg and support a patient in a desired position.

7. The modular patient positioning system of claim 3, further including
a leg positioner including a boot portion adapted to receive a foot and a lower portion of a leg; the boot portion further comprising pegs for being received in the at least one of said pegboard.

8. The modular patient positioning system of claim 3, wherein the second pegboard is L-shaped having a narrow portion and a wide portion and is configured to be removably connected to said first pegboard by said hinge such that the wide portion and the narrow portion of the second pegboard can be selectively connected to said first pegboard by said hinge.
